(19) United States
(12) Patent Application Publication
(10) Pub. No.: US 2025/0213621 A1
(43) Pub. Date: Jul. 3, 2025

(54) CARDIOPULMONARY PROGENITOR EXOSOMES, PREPARATION METHOD AND APPLICATION THEREOF

(71) Applicant: Guangzhou Medical University, Guangzhou (CN)

(72) Inventors: Xi-Yong Yu, Guangzhou (CN); Yingying Xiao, Guangzhou (CN); Luoxing Xia, Guangzhou (CN); Jianfeng Qin, Guangzhou (CN); Li Wei, Guangzhou (CN)

(21) Appl. No.: 18/736,487

(22) Filed: Jun. 6, 2024

(30) Foreign Application Priority Data
Dec. 28, 2023 (CN) 202311836685.9

(51) Int. Cl. A61K 35/34; A61K 35/42; A61P 9/10; C12N 5/00; C12N 5/071; C12N 5/077

(52) U.S. Cl. CPC A61K 35/34; A61K 35/42; A61P 9/10; C12N 5/0018; C12N 5/0657; C12N 5/0689; C12N 2500/32; C12N 2500/44; C12N 2500/84; C12N 2500/99; C12N 2501/115; C12N 2501/415; C12N 2525/00

(57) ABSTRACT

Cardiopulmonary progenitor exosomes and a preparation method thereof are provided. A culture medium of the cardiopulmonary progenitors is prepared by adopting a preparation method developed by an inventor aiming at the cardiopulmonary progenitors, the supernatant is taken, and the cardiopulmonary progenitor exosomes are isolated and extracted by ultracentrifugation. The applications of the cardiopulmonary progenitor exosomes in reducing the area of cardiac necrosis and fibrosis, promoting the improvement of cardiac function, the proliferation of cardiomyocytes and the angiogenesis of injured hearts are provided, which indicates that they have great potential in preventing and treating cardiovascular diseases.

### CROSS-REFERENCE TO RELATED APPLICATION

This application claims priority to Chinese Patent Application No. 202311836685.9, filed Dec. 28, 2023, which is herein incorporated by reference in its entirety.

### TECHNICAL FIELD

The disclosure relates to the field of biotechnologies, and more particularly to cardiopulmonary progenitor exosomes, a preparation method and an application thereof.

### BACKGROUND

Progenitors (also referred to as progenitor cells) refer to undifferentiated pluripotent or specialized stem cells, usually found in adult tissues. The progenitors can be mobilized and activated to proliferate in large numbers and migrate to injured areas, and then differentiate and replace injured tissues, which has the function of helping to repair and regenerate tissues and organs. Therefore, scientists are trying to cultivate the progenitors and transplant them into patients to treat various degenerative diseases.

The inventor's team of the disclosure has previously developed a preparation method for cardiopulmonary progenitors, which can stably obtain the cardiopulmonary progenitors. However, the effects of the cardiopulmonary progenitors on the injured heart or their mechanisms have not been thoroughly investigated, let alone the preparation of cardiopulmonary progenitor exosomes, and it is even more unknown whether the cardiopulmonary progenitor exosomes can be biologically active and what kind of bioactivity they have.

### SUMMARY

In view of this, purposes of the disclosure are to provide cardiopulmonary progenitor exosomes, and its preparation method and its application in promoting the proliferation of cardiomyocytes.

Technical solutions for achieving the above purposes include the following.

In a first aspect of the disclosure, a preparation method of cardiopulmonary progenitor exosomes is provided.

In some embodiments, steps of the preparation method include the follow steps:
S1, preparing cardiopulmonary progenitors and culturing the cardiopulmonary progenitors in a culture medium of the cardiopulmonary progenitors; and
S2, separating the culture medium of the cardiopulmonary progenitors cultured in the step S1 by ultracentrifugation to obtain the cardiopulmonary progenitor exosomes.

In some embodiments, the step S2 specifically includes:
centrifuging the culture solution of the cardiopulmonary progenitors cultured in step S1 at 290-310 gravitational acceleration (g) for 8-12 minutes (min), at 1,990-2,010 g for 8-12 min, and at 9,900-10,100 g for 25-35 min to obtain a first centrifuged mixture, then taking a first supernatant from the first centrifuged mixture, centrifuging the first supernatant at 99,900-100, 100 g for 62-78 min to obtain a second centrifuged mixture, removing a second supernatant from the second centrifuged mixture to obtain a precipitate, and resuspending the precipitate with phosphate-buffered saline (PBS), so as to obtain the cardiopulmonary progenitor exosomes.

In some embodiments, the step S1 includes the following:
SA) isolating an area where the cardiopulmonary progenitors are located from a mammalian embryo on a 9.5^{th} day;
SB) digesting the area where the cardiorespiratory progenitors are located in the step SA) with a digestion solution to obtain a digested mixture, centrifuging the digested mixture and then collecting the cardiorespiratory progenitors;
SC) performing hanging drop culture on the cardiorespiratory progenitors for 46-52 hours by inducing embryoids to obtain a first suspension, with 1,970-2,030 cells in each 15 microliters (μL) of the first suspension;
SD) performing suspension culture on the first suspension, including:
   culturing the first suspension in a differentiation medium for 22-26 hours (h) to obtain a first cultured product, then culturing the first cultured product in a basic medium for 22-26 h to obtain a second cultured product, digesting the second cultured product into a single-cell suspension, and inoculating the single-cell suspension on a Petri dish coated with gelatin to obtain inoculated cells; and
SE) continuing to culture the inoculated cells with an ABC medium, changing the ABC medium every two days, and performing passage culture until a cell confluence is more than 90%.

In some embodiments, the passage culture is performed at a ratio of 1:3-4.

In some embodiments, the culture medium of the cardiopulmonary progenitors is ABC medium.

In some embodiments, the ABC culture medium includes 1.5-2.5% B-27 without vitamin A, 1.5-2.5 millimoles per liter (mM) L-glutamine, 0.8-1.2% nonessential amino acids, 0.08-0.12 mM β-mercaptoethanol, 0.8-1.2 μM A83-01, 45-55 nanograms per milliliter (ng/ml) basic fibroblast growth factor (bFGF), 10-14 μM CHIR-99021, and 2.5-5.0% human platelet lysates (HPLs).

In some embodiments, the ABC culture medium includes 1.9-2.1% B-27 without vitamin A, 1.9-2.1 mM L-glutamine, 0.9-1.1% nonessential amino acids, 0.09-0.11 mM β-mercaptoethanol, 0.9-1.1 μM A83-01, 49.5-50.5 ng/mL bFGF, 11-13 μM CHIR-99021, and 2.4-2.6% HPLs.

In a second aspect of the disclosure, cardiopulmonary progenitor exosomes are provided.

In some embodiments, a particle size of the cardiopulmonary progenitor exosomes is in a range of 131-142.8 nanometers (nm).

According to the disclosure, the cardiopulmonary progenitor exosomes are prepared by selecting ultracentrifugation method, which are isolated from many other extracellular vesicle structures distributed in a complex humoral environment, and their biological activities are studied, so as to obtain relevant valuable medical applications.

In a third aspect of the disclosure, an application of the aforementioned cardiopulmonary progenitor exosomes or the preparation method of the aforementioned cardiopulmonary progenitor exosomes in preparing medicines for preventing and treating cardiovascular diseases in mammals is provided.

In a fourth aspect of the disclosure, an application of the aforementioned cardiopulmonary progenitor exosomes or the preparation method of the aforementioned cardiopulmonary progenitor exosomes in improving cardiac functions of mammals is provided.

In some embodiments, the cardiovascular disease is myocardial infarction.

In some embodiments, the above application is to promote the proliferation of heart tissue cells of the mammals.

In some embodiments, the cardiac tissue cells include at least one of cardiomyocytes, pericytes, myofibroblasts, smooth muscle cells and endothelial cells.

In some embodiments, the above application is to promote cardiovascular generation in the mammals.

In some embodiments, the above application is to increase cardiovascular density in the mammals.

In some of these embodiments, the above application is to reduce an area of at least one of cardiac necrosis and fibrosis in the mammals.

In some embodiments, the above application is to improve the left ventricular ejection fraction (LVEF) and/or the left ventricular fractional shortening (LVFS) in the mammals.

Compared with the related art, the disclosure has the following beneficial effects.

According to the disclosure, it is found that the cardiopulmonary progenitor exosomes have a remarkable effect on promoting the proliferation of cardiac tissue cells, and also have a good performance in improving cardiac function, reducing the area of cardiac necrosis and fibrosis and promoting the angiogenesis of injured hearts. It indicates that the cardiopulmonary progenitor exosomes have great potential in the prevention and treatment of cardiovascular diseases, such as repairing infarcted myocardium and promoting the regeneration of cardiac fibroblasts. Moreover, it is found that the function of cardiopulmonary progenitors (CPPs) to improve cardiac function is not achieved through the proliferation or differentiation of CPPs themselves, but through the paracrine pathway, especially secreted exosomes. The disclosure provides the cardiopulmonary progenitor exosomes, the preparation method thereof, and the applications thereof in preparing medicines for preventing and treating cardiovascular diseases of animals, such as myocardial infarction, and in preparing medicines for improving the cardiac function of mammals.

### BRIEF DESCRIPTION OF DRAWINGS

FIG. 1A illustrates a schematic diagram of markers Is11, Wnt2 and Gli1 of CPPs detected by immunofluorescence staining, where a scale of the upper row of pictures is 100 micrometers (μm) and a scale of the lower row of pictures is 50 μm.

FIG. 1B illustrates a schematic diagram of detection results of the markers Isl1, Wnt2 and Gli1 of the CPPs detected by flow cytometry.

FIG. 2 illustrates a schematic diagram of differentiation processes of the CPPs in vitro. Specifically, the CPPs differentiated into cardiomyocytes (a), endothelial cells (b), fibroblasts (c), smooth muscle cells (d) and alveolar epithelial cells (e) under different induction conditions.

FIG. 3 illustrates an extraction process of cardiopulmonary progenitor exosomes (CPPs-Exo).

FIGS. 4A-4C illustrate identification results of characteristics of the CPPs-Exo. Specifically, FIG. 4A illustrates analysis results of particle size and concentration of the CPPs-Exo by nanoparticle tracking, FIG. 4B illustrates an analysis picture of the CPPs-Exo with the typical cup-shaped appearance of microvesicles observed by transmission electron microscopy, and FIG. 4C illustrates exosome markers Alix and Hsp70 detected by Western blotting.

FIGS. 5A-5C illustrate teratoma formation risk assessment and karyotype analysis results of the CPPs. Specifically, FIG. 5A illustrates the grouping of nude mice subcutaneously injected with the CPPs and mouse embryonic stem cells (mESCs), FIG. 5B illustrates the teratoma formation after 3 months, and FIG. 5C illustrates the karyotype analysis result of the CPPs.

FIGS. 6A-6C illustrate representative pictures of left ventricular short-axis ultrasound results of myocardial infarction (MI) mice after grouping treatment and its data analysis results (including LVEF shown in FIG. 6B and LVFS shown in FIG. 6C); and the CPPs promote the improvement of cardiac function in MI mice through their exosomes.

FIG. 7A illustrates results of Masson staining results of cardiac tissue sections of the MI mice after grouping treatment.

FIG. 7B illustrates results of Sirus red staining of the cardiac tissue sections of the MI mice after the grouping treatment.

FIG. 7C illustrates analysis results of infarct size (also referred to as necrosis area) of the cardiac tissue sections of the MI mice after the grouping treatment. CPPs reduce the cardiac infarct size of the MI mice through their exosomes.

FIG. 7D illustrates data analysis results of data on proportion of fibrosis (i.e., fibrotic area/LV area). CPPs reduce the area of cardiac fibrosis in the MI mice through their exosomes.

FIG. 8A illustrates staining results of a smooth muscle marker alpha smooth muscle actin (α-SMA) of the MI mice after the group treatment (scale: 500 μm).

FIG. 8B illustrates a vascular density analysis of the MI mice after the group treatment. CPPs promotes angiogenesis of injured heart through their exosomes.

FIG. 9 illustrates staining results of proliferating cardiomyocyte markers cTnT and Ki67 in the MI mice after the grouping treatment. The ascending scale of each group is 500 μm, and the descending scale of each group is 250 μm. CPPs promote the proliferation of cardiomyocytes through their exosomes.

FIG. 10 illustrates analysis results of proportion of proliferation of cardiomyocytes in MI mice after grouping treatment, in which the effect of promoting the proliferation of the cardiomyocytes in MI+CPPs-Exo group is significantly better than that in MI+CPPs group.

FIG. 11 illustrates a plot of mouse cardiac sections hybridized with a Y chromosome probe at a scale of 20 μm.

### DETAILED DESCRIPTION OF EMBODIMENTS

In order to facilitate the understanding of the disclosure, a more comprehensive description of the disclosure is given below. The disclosure can be implemented in many different forms and is not limited to the embodiments described herein. On the contrary, these embodiments are provided to make the understanding of the disclosure more thorough and comprehensive.

In the following embodiments, the experimental methods without specific conditions are generally in accordance with the conventional conditions, for example, the fourth edition of Molecular Cloning: A Laboratory Manual edited by Green and Sambrook has been published in 2013; or as recommended by the manufacturer. Various common chemical reagents used in the embodiments are all commercially available products.

Unless otherwise defined, all technical and scientific terms used herein have the same meaning as commonly understood by those skilled in the art to which the disclosure belongs. The terminology used in the description of the disclosure is for the purpose of describing particular embodiments only and is not intended to be limiting of the disclosure. The term “and/or” as used herein includes any and all combinations of one or more of the associated listed items.

Myocardial infarction refers to ischemic necrosis of myocardium. On the basis of coronary artery disease, the blood flow of coronary artery is sharply reduced or interrupted, resulting in severe and persistent acute ischemia of the corresponding myocardium, which eventually leads to ischemic necrosis of the myocardium.

The disclosure will be further described in detail with specific embodiments.

### Embodiment 1

### 1. Cardiopulmonary Progenitors (CPPs, Also Referred to as Cardiopulmonary Progenitor Cells) are Prepared, and the Method and Results are as Follows.

Under a dissecting microscope, the head and tail of the C57BL/6 mouse (GemPharmatech Co., Ltd) embryos at embryonic day 9.5 (E9.5) are removed, and then the CPPs region is isolated and digested with a digestion solution (0.04% trypsin and 0.05% collagenase IV) at 37° C. for 10 min, and slightly reversed several times in the middle. After the digestion is terminated with FBS-containing medium (Roswell Park Memorial Institute 1640 medium, abbreviated as RPMI 1640 medium), centrifugation at 200×g is performed for 5 min. After the collected cells are counted, the collected cells are cultured by the embryonic bodies (EB) method (for example, referring to: “Isolation and Functional Characterization of Pluripotent Stem Cell-Derived Cardiac Progenitor Cells”, Curr Protoc Stem Cell Biol. 2010 September; DOI: 10.1002/9780470151808.sc01f10s14; Page 4, Steps 5-6). Hanging drop culture is performed, with a volume of each drop of suspension being 15 μL and the number of cells being 2000. After two days of the hanging drop culture, suspension culture is performed, cultured 1 day with a basic medium supplemented with 12 μM CHIR99021, followed by a basic medium (RPMI 1640 added with 2% B-27 without insulin, 2 mM L-glutamine, 1% NEEA, 1% penicillin/streptomycin, 0.1 mM β-mercaptoethanol) for another day. The cells are then digested into a single-cell suspension, plated on a Petri dish coated with 0.2% gelatin gum, and continued to be cultured with an ABC medium (Table 1). The medium is changed every 2 days until the cell confluency is greater than 90%, and the cells are passaged at 1:3. The specific culture process is shown in FIG. 3. The composition (volume ratio) of the ABC medium is as follows:

TABLE 1
Reagents and concentrations added to
ABC medium in DMEM/F12 basic medium.

| Reagents | Final concentration used | |
|---|---|---|
| B-27, without vitamin A | 2% | |
| L-glutamine | 2 | mM |
| nonessential amino-acids (NEEA) | 1% | |
| β-mercaptoethanol | 0.1 | mM |
| A83-01 (TGF-β type I receptor inhibitor) | 1 | μM |
| bFGF | 50 | ng/mL |
| CHIR-99021 | 12 | μM |

| | |
|---|---|
| Human platelet lysate | 2.5% |
| (purchased from PL BioSience) | |

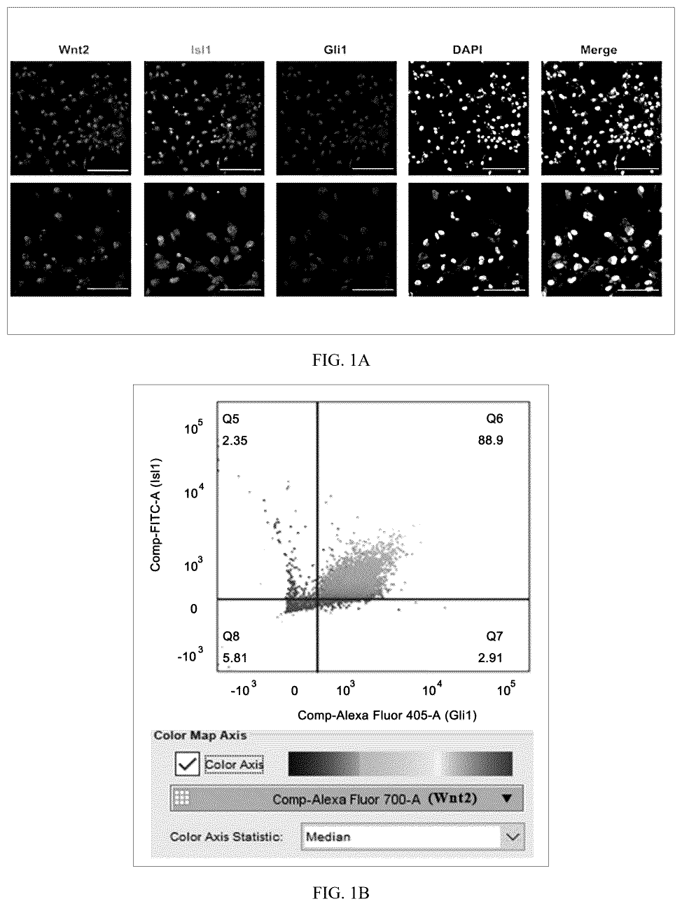

The CPPs can be obtained by the above passage. Immunofluorescence and flow cytometry are used to verify that high purity CPPs expressing Isl1, Wnt2 and Gli1 are obtained by the above preparation method. The results can be seen in FIG. 1.

### 2. Identification of Differentiation Ability of CPPs

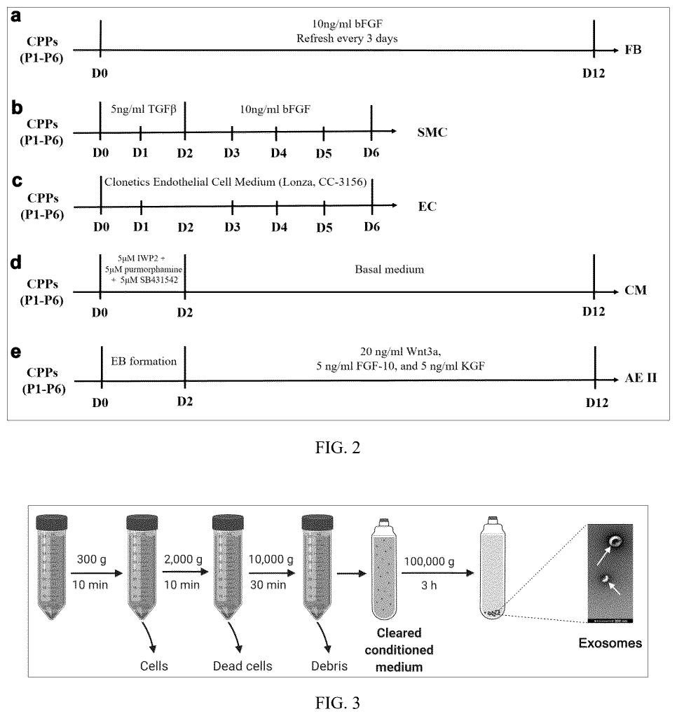

CPPs are induced to differentiate in vitro according to the induction method shown in FIG. 2 (a-e), and the cells are collected at day 6 or 12 for post-differentiation identification by flow cytometry, immunofluorescence, and reverse transcription quantitative polymerase chain reaction (RT-qPCR), respectively. The results determine that the CPPs can differentiate into fibroblasts, cardiomyocytes, endothelial cells, smooth muscle cells and alveolar epithelial cells, and that the percentages of cells that differentiated successfully in this embodiment are 74.7%, 75.4%, 92.7%, 88.5% and 47.9% respectively. Therefore, the CPPs obtained by isolation and culture in the disclosure have the ability to differentiate into cardiomyocytes, fibroblasts, endothelial cells, smooth muscle cells and alveolar epithelial cells in vitro.

### 3. The Method and Results for Preparing Exosomes of the CPPs are as Follows.

Under the condition that the centrifugation process is kept at 4° C., the exosomes in the culture solution (supernatant) of the CPPs are isolated and extracted by ultracentrifugation: centrifugation at 300 g for 10 min to remove dead cells; centrifugation at 2,000 g for 10 min to remove cell debris; centrifugation at 10,000 g for 30 min to take the supernatant, and centrifugation at 100,000 g for 3 h to remove the supernatant, and the exosomes are resuspended with PBS, and stored separately at −80° C.

### 4. The Exosomes of the CPPs are Identified by Western Blotting.

### (1) Extraction of Total Cellular Protein

1) An ice box is prepared, the cells are collected whose proteins to be extracted from a 6-well plate, the medium in the well plate is discarded, and the well plate is rinsed with PBS buffer twice. 100 μL of radio-immunoprecipitation assay (RIPA) lysis buffer containing phenylmethylsulfonyl fluoride (PMSF) is added into the well plate, and then the cells are lysed on ice for half an hour.
2) After cell lysis, the cells are scraped off with a clean cell scraper, the cell lysate is concentrated on one side of the well plate with the scraper, followed by transferring the protein suspension to a 1.5 mL Eppendorf (EP) tube by aspiration using a pipette gun. In order to fully release the proteins in the cells, the protein suspension is ultrasonicated with an ultrasonic cell disruptor for 80 watts (W) and 30 seconds(s), with 5 s on and 2 s off.
3) After ultrasound, all samples are balanced, centrifuged at 12,000 revolutions per minute (rpm) at 4° C. for 20 min. The supernatant is carefully transferred to a new 1.5 mL EP tube, which is the extracted total cellular protein.

### (2) Determination of Protein Concentration and Protein Denaturation

1) According to the purchased bicinchoninic acid (BCA) protein concentration assay kit, 50× protein standard is configured to obtain a 0.5 mg/mL BSA protein standard.
2) According to Table 2, PBS and BSA are added to prepare a protein concentration standard curve.

TABLE 2
Configuration of protein standard

| 0.5 mg/mL standard (μL) | PBS buffer (μL) | Concentration (mg/mL) |
|---|---|---|
| 0 | 20 | 0 |
| 1 | 19 | 0.025 |
| 2 | 18 | 0.05 |
| 4 | 16 | 0.1 |
| 8 | 12 | 0.2 |
| 12 | 8 | 0.3 |
| 16 | 4 | 0.4 |
| 20 | 0 | 0.5 |

3) Protein samples and standards are added into a 96-well plate according to the kit instructions, followed by the addition of BCA working solution and incubation at 37° C. for 30 min.
4) The reaction plate is measured for absorbance at a wavelength of A562 using a microplate reader. The protein standard curve is created based on the absorbance values, and a linear regression equation is derived, and then the sample concentration is calculated.
5) 5× loading buffer and PBS are added according to the calculated protein concentration, so that the protein mass of each sample was 30-50 μg.
6) Subsequently, all samples are heated in a 95° C.-water bath for 5 min to denature the proteins, and then stored at −80° C. for later use.

### (3) Sodium Dodecyl Sulfate Polyacrylamide Gel Electrophoresis (SDS-PAGE)

1) According to Table 3, separator gel (also referred to as resolving gel) and concentrated gel (also referred to as stacking gel) are configured.

TABLE 3
SDS-PAGE gel formula

| | | | Stacking |
|---|---|---|---|
| | Resolving gel (30 mL) | | gel (10 mL) |
| Required components | 10% | 8% | 5% |
| Distilled water | 12.2 | 14.2 | 5.8 |
| 30% Acr-Bis (29:1) | 10.0 | 8.0 | 1.7 |
| Lower gel buffer (4×) | 7.5 | 7.5 | — |
| Upper gel buffer (4×) | — | — | 2.5 |
| 10% ammonium persulfate | 0.4 | 0.4 | 0.2 |
| N,N,N′,N′- | 0.012 | 0.012 | 0.01 |
| Tetramethylethylenediamine | | | |
| (TEMED) | | | |

2) Sample loading: the solidified gel is put into the electrophoresis tank as required, the prepared 1× electrophoresis transfer buffer, the prepared protein samples and electrophoresis marker are added according to the group order, and labeled.
3) Electrophoresis: 80 volts (V) constant voltage electrophoresis is performed for about 30 min, then the voltage is adjusted to 100 V, and electrophoresis is performed 90-100 min.
4) Membrane transfer: after the electrophoresis is complete, the glass plate is carefully removed. The polyvinylidene fluoride (PVDF) membrane, along with the gel and a sponge that have been soaked in methanol, are assembled into a sandwich configuration using clips. This sandwich is then placed into the membrane transfer apparatus. 1 liter (L) of transfer buffer is added to the tank, and a constant current of 200 milliamperes (mA) is set for the transfer. The transfer is performed for a duration of 90-120 min.
5) Blocking: at the end of the membrane transfer process, the PVDF membrane is removed and placed in a Western blotting (WB) blocking solution. The membrane is positioned facing upwards and agitated on a shaker at a speed of 55 rpm to ensure even distribution of the solution. The membrane is then incubated at room temperature for 1-2 h to allow for proper blocking.
6) Incubation of primary antibodies: after blocking is complete, the excess blocking solution with Tris-buffered saline with Tween (TBST) buffer is washed off. Based on the molecular weight of the target protein, the PVDF membrane is cut to the appropriate size and then placed into a container with the corresponding primary antibodies (Gli1, Isl1, Wnt2, Tbx5, GAPDH). The membrane is incubated with the primary antibodies overnight at 4° C.
7) Incubation of secondary antibodies: the following day, the PVDF membrane is taken out and the primary antibodies are recovered. The PVDF membrane is cleaned with the TBST buffer at high speed for three cycles, with each cycle lasting 10 min. Subsequently, the working solutions of anti-rabbit and anti-mouse secondary antibodies corresponding to the primary antibodies are added, and incubated for 1 h on a shaker at room temperature at 55 rpm.
8) Enhanced chemiluminescence (ECL) detection: after the second antibody incubation, the PVDF membrane is washed with the TBST buffer at 120 rpm for three cycles, with each cycle lasting 10 min. Subsequently, ECL chemiluminescence reagent is added to the front of the PVDF membrane, which is put into an ultra-sensitive multifunctional imager for imaging, and the image is saved for subsequent analysis.

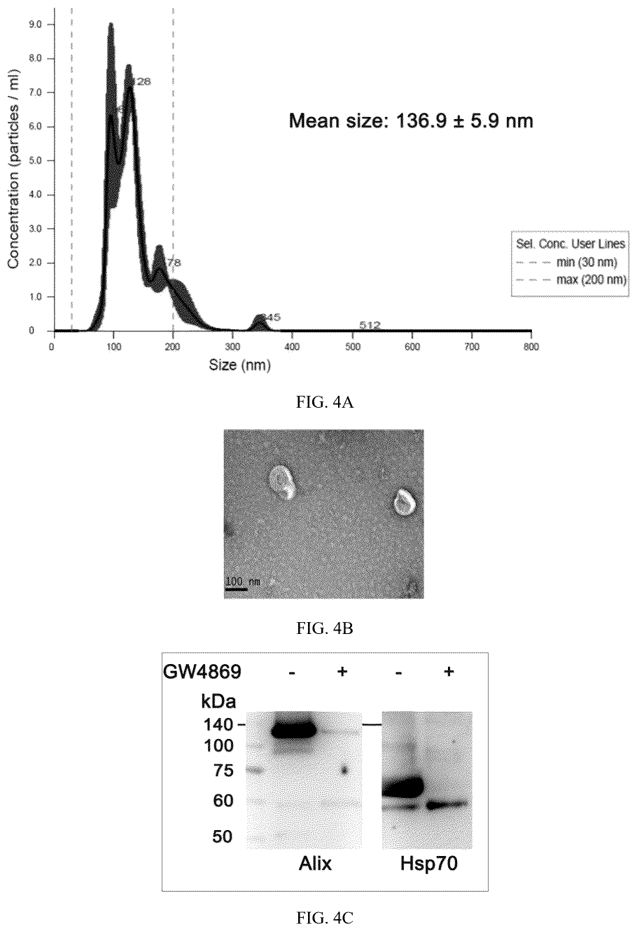

The extraction process of CPPs exosomes (CPPs-Exo) is shown in FIG. 3, the exosomes are extracted from the supernatant of CPPs culture solution by ultracentrifugation kept at 4° C. The particle size of the exosomes is then determined to be 136.9±5.9 nm (see FIG. 4A) and the concentration to be 4.37×10⁹/mL using nanoparticle tracking analysis (NTA). The morphology of exosomes is detected by transmission electron microscopy and identified as a typical cup-shaped appearance of microvesicles (see FIG. 4B), and the surface markers Alix and Hsp70 of exosomes are identified by Western blotting (see FIG. 4C). These results are consistent with the basic characteristics of exosomes, indicating that the extracted products are indeed exosomes.

### Embodiment 2

The function of the CPPs-Exo is explored, it is found that CPPs promote myocardial repair after injury through their exosomes.

### 1. Tumorigenicity Test of CPPs

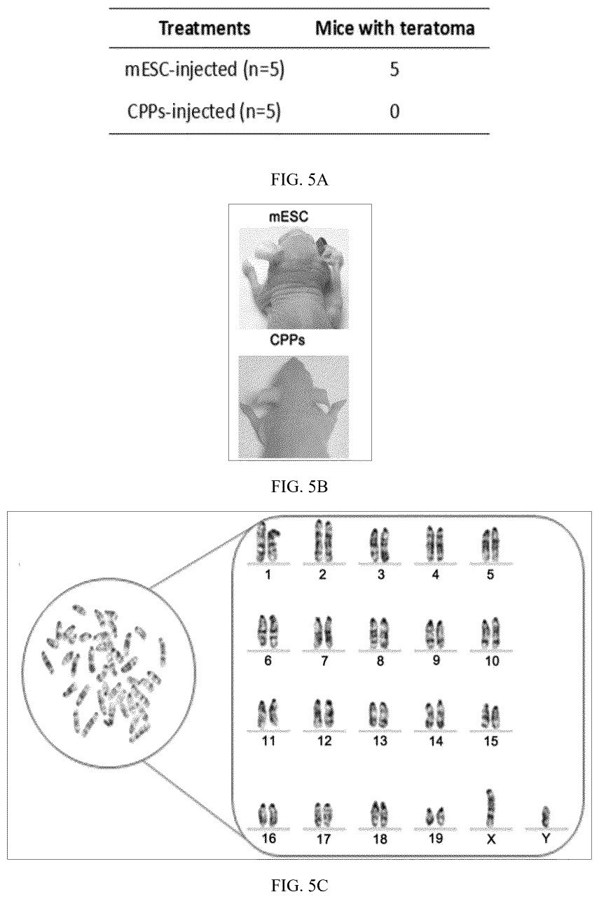

10 mice are randomly divided into two groups (as shown in FIG. 5A) for subcutaneous tumor transplantation experiment. One group of nude mice is injected with mouse embryonic stem cells (mESC-injected) and the other group of nude mice is injected with cardiopulmonary progenitors (CPPs-injected) prepared by the above method to evaluate the tumorigenicity of CPPs. The teratoma formation after 3 months is shown in FIG. 5B, and no teratoma is observed after CPPs injection. Therefore, CPPs can be used for transplantation. FIG. 5C is a karyotype analysis result of injected CPPs. It can be seen that the isolated and cultured CPPs contain Y chromosome. Therefore, female mice are selected to model MI model, so as to track the transdifferentiation of CPPs in vivo by Y chromosome probe hybridization.

The following MI mice are all females.

### 2. Effects of CPPs-Exo on Cardiac Function in MI Mice.

Mice with myocardial infarction (MI) are modeled and randomly divided into two groups, and treated as follows:
SHAM group: sham operation group;
MI+PBS group: intramyocardial injection of PBS after MI;
MI+CPPs group: intramyocardial injection of CPPs after MI;
MI+CPPs (GW4869) group: intramyocardial injection of CPPs treated with inhibitor GW4869 after MI;
MI+CPPs Fragments group: intramyocardial injection of cell fragments of CPPs after MI; and
MI+CPPs-Exo group: intramyocardial injection of CPPs-Exo after MI.

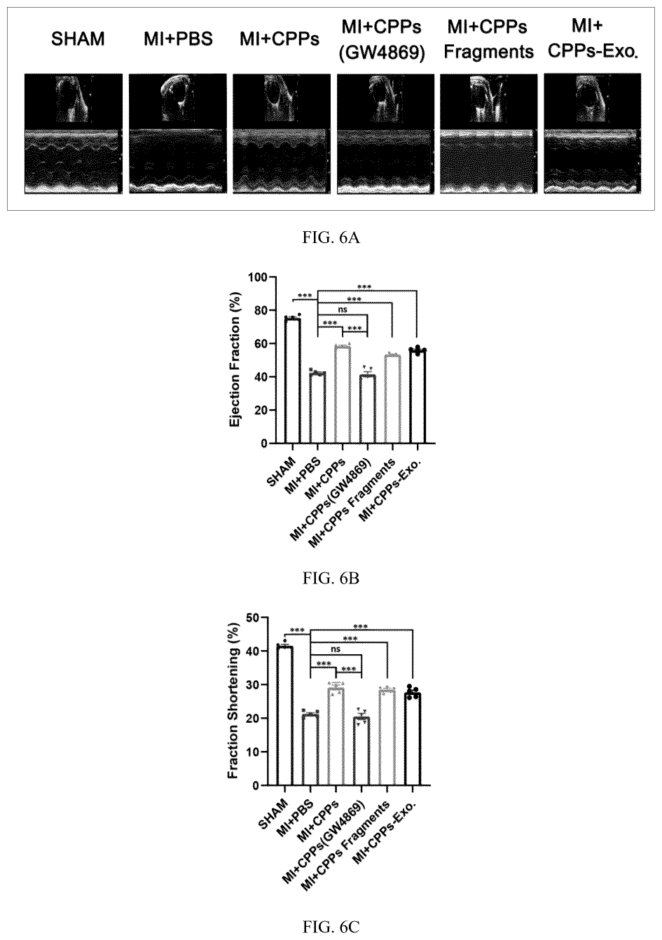

FIG. 6A illustrates results of left ventricular short-axis ultrasound after 42 days of modeling in MI mice. FIGS. 6B-6C illustrate that: compared with the MI+PBS group, the LVEF and LVFS in the MI+CPPs group are significantly increased, indicating that the cardiac function has been repaired; and compared with the MI+CPPs group, the LVEF and LVFS in the MI+CPPs (GW4869) group are significantly decreased, indicating that inhibiting the secretion of the CPPs-Exo significantly inhibits the cardiac repair function of CPPs. The CPPs-Exo promotes the improvement of cardiac function in MI mice.

### 3. Effects of CPPs-Exo on Cardiomyocytes

The following data are presented as mean±standard error of the mean (SEM), with 5 biological replicates in each group, with ns P>0.05 and ***P<0.001 (t test).

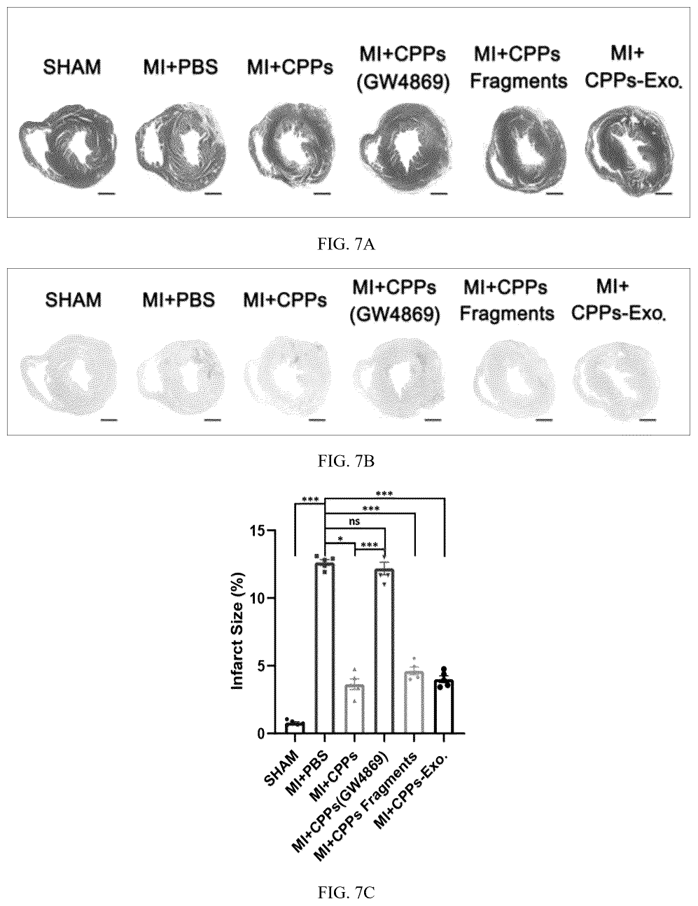

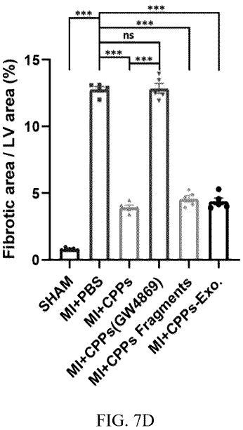

FIGS. 7A-7D illustrate results of Masson staining, Sirus red staining, and digital analysis results of cardiomyocytes of MI mice respectively after the above grouping treatment. The results show that the infarct sizes (12.37% and 12.12% respectively) and the fibrosis sizes (12.56% respectively) of the MI+PBS group and the MI+CPPs (GW4869) group are significantly different from those of SHAM group, suggesting that the cardiac function of the two groups deteriorated. However, the measured values of the MI+CPPs group, the MI+CPPs Fragments group and the MI+CPPs-Exo group are all lower than 5%, which is significantly lower than that of the MI+PBS group. This suggests that CPPs and CPPs-Exo can reduce scar formation. CPPs-Exo can reduce the area of cardiac necrosis and fibrosis in the MI mice.

All the above results suggest that the MI-CPP group, the MI-CPPs Fragments group and the MI-CPPs-Exo group can promote the improvement of cardiac function, and the infarct size and fibrosis degree of the heart are significantly reduced, while the CPPs treated with GW4869 can inhibit the synthesis and release of their exosomes, thereby reversing the repair effect of the CPPs. These results prove that CPPs-Exo can improve the function of injured heart.

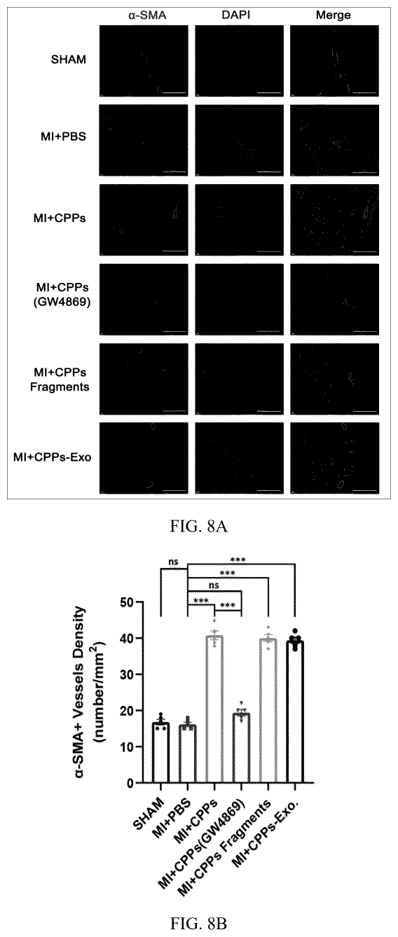

In order to evaluate the effect of CPPs on angiogenesis and cardiomyocyte proliferation, the vascular smooth muscle cell marker α-SMA (see FIG. 8A, scale 500 μm) and the proliferating cell marker Ki67 (see FIG. 8B) are used to perform immunofluorescence staining of cardiac tissue sections. The results refer to FIG. 8B, in the MI+CPPs group, the MI+CPPs Fragments group and the MI+CPPs-Exo group, the α-SMA+ vessels density is increased to nearly 40 per square millimeter (/mm²), while in the SHAM group, the MI+PBS group, and the MI+CPPs (GW4869) group, the α-SMA+ vessels density is lower than 20/mm². CPPs-Exo promotes angiogenesis of the injured heart.

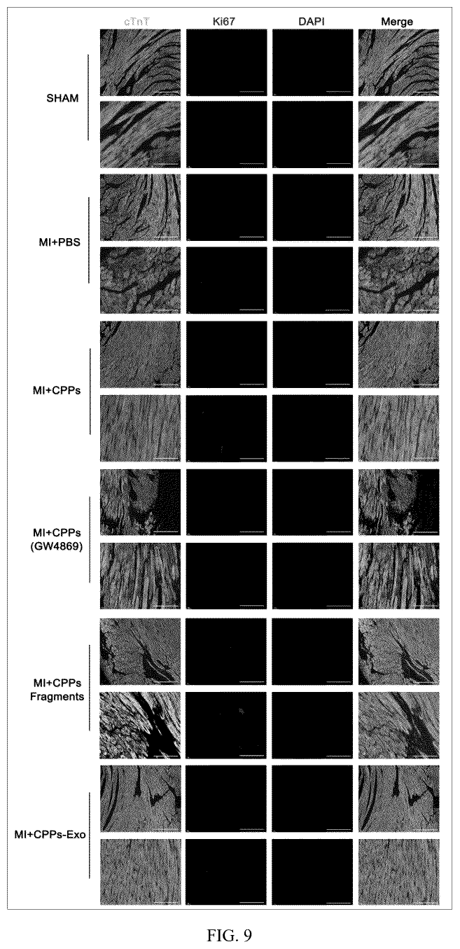

FIG. 9 illustrates staining results of proliferating cardiomyocyte markers cTnT and Ki67 in the MI mice after the grouping treatment. The ascending scale of each group is 500 μm, and the descending scale of each group is 250 μm. CPPs promote the proliferation of cardiomyocytes in MI mice through their exosomes.

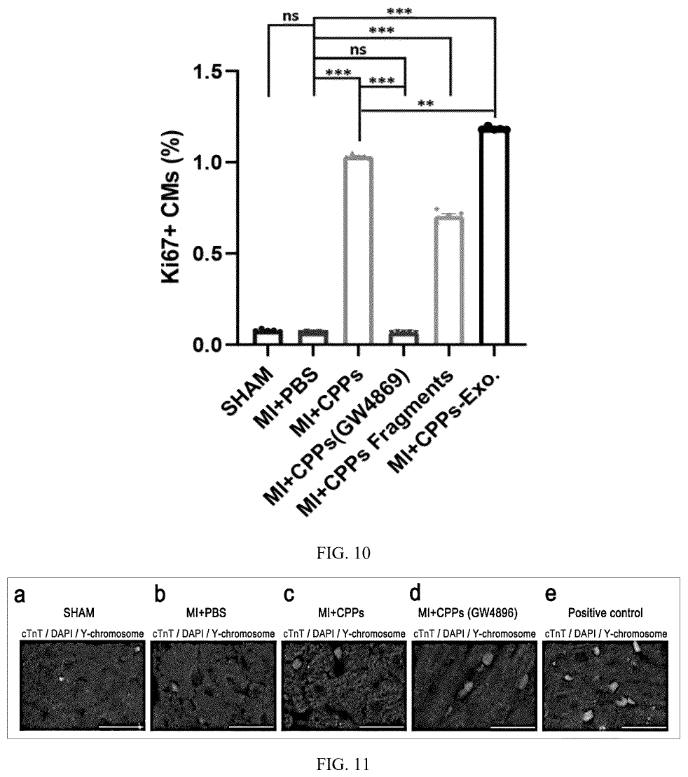

As shown in FIG. 10, in the MI+CPPs group, the MI+CPPs Fragments group and the MI+CPPs-Exo group, the proportions of proliferating cardiomyocytes (identified by cTnT and Ki67 antibody co-staining) are significantly higher (1.03%, 0.71% and 1.18% respectively), while in the SHAM group, the MI+PBS group and the MI+CPPs (GW4869) group, only about 0.06%, indicating that CPPs can promote the proliferation of cardiomyocytes. However, if GW4869 is used to inhibit the secretion of exosomes, the effect of promoting proliferation will be reversed. Therefore, CPPs promote the proliferation of cardiomyocytes through their secreted exosomes. The effect of the MI+CPPs-Exo group on promoting cardiomyocyte proliferation is significantly better than that of the MI+CPPs group (the proportions of Ki67 positive cardiomyocytes are 1.03% and 1.18% respectively). Therefore, the results show that CPPs-Exo promotes the proliferation of cardiomyocytes in the MI mice.

MI mice are divided into a. a SHAM group, b. a post-MI intramyocardial injection of PBS group, c. a post-MI intramyocardial injection of CPPs group, d. a post-MI intramyocardial injection of GW4896-treated CPPs group, and e. a positive group (male mice); and Y chromosome probes are used to hybridize cardiac sections of the mice after group treatment, and the results are compared with those of the positive control group. As can be seen by the hybridization of cardiac sections of the mice after group treatment with the Y-chromosome probes shown in parts a-e (scale: 20 μm) of FIG. 11, which is shown in a-e (scale 20 μm) of FIG. 11, no signal is detected in the hearts of MI female mice after 42 days of CPPs injection, indicating that CPPs injected into the myocardium do not survive or transdifferentiate into other cells 42 days after CPPs injection. Therefore, the above-mentioned function of promoting cardiac function improvement brought by CPPs is not achieved by the proliferation or differentiation of CPPs themselves, but by paracrine pathways, such as exosomes secreted by CPPs.

The above-mentioned embodiments only express several embodiments of the disclosure, and their descriptions are more specific and detailed, but they cannot be understood as limiting the scope of disclosure patents. It should be pointed out that for those skilled in the art, without departing from the concept of the disclosure, a number of variations and improvements can be made, which are within the scope of protection of the disclosure. Therefore, the scope of protection of the patent of this disclosure should be based on the appended claims.

1. A preparation method of cardiopulmonary progenitor exosomes, comprising:
S1, preparing cardiopulmonary progenitors and culturing the cardiopulmonary progenitors in a culture medium of the cardiopulmonary progenitors; and
S2, centrifuging the culture medium of the cardiopulmonary progenitors cultured in the step S1 at 290-310 gravitational acceleration (g) for 8-12 minutes (min), at 1,990-2,010 g for 8-12 min, and at 9,900-10,100 g for 25-35 min to obtain a first centrifuged mixture, then taking a first supernatant from the first centrifuged mixture, centrifuging the first supernatant at 99,900-100,100 g for 62-78 min to obtain a second centrifuged mixture, removing a second supernatant from the second centrifuged mixture to obtain a precipitate, and resuspending the precipitate with phosphate-buffered saline (PBS), so as to obtain the cardiopulmonary progenitor exosomes;
wherein the culture medium of the cardiopulmonary progenitors is ABC medium; the ABC culture medium comprises 1.5-2.5 millimoles per liter (mM) L-glutamine, 0.8-1.2% nonessential amino acids, 0.08-0.12 mM β-mercaptoethanol, 0.8-1.2 μM A83-01, 45-55 nanograms per milliliter (ng/mL) basic fibroblast growth factor (bFGF), 10-14 μM CHIR-99021, and 2.5-5.0% human platelet lysates (HPLs).

2. The preparation method as claimed in claim 1, wherein the step S2 comprises:
centrifuging the culture medium of the cardiopulmonary progenitors cultured in the step S1 at 300 g for 10 min, at 2,000 g for 10 min, and at 10,000 g for 30 min to obtain the first centrifuged mixture, then taking the first supernatant from the first centrifuged mixture, centrifuging the first supernatant at 100,000 g for 70 min to obtain the second centrifuged mixture, removing the second supernatant from the second centrifuged mixture to obtain the precipitate, and resuspending the precipitate with the PBS, so as to obtain the cardiopulmonary progenitor exosomes.

3. The preparation method as claimed in claim 1, wherein the ABC culture medium comprises 2 mM L-glutamine, 1% nonessential amino acids, 0.1 mM β-mercaptoethanol, 1 μM A83-01, 50 ng/mL bFGF, 12 μM CHIR-99021, and 2.5% HPLs.

4. The preparation method as claimed in claim 1, wherein the step S1 comprises:
SA) isolating an area where the cardiopulmonary progenitors are located from a mouse embryo on a 9.5^{th} day;
SB) digesting the area where the cardiorespiratory progenitors are located in the step SA) with a digestion solution to obtain a digested mixture, centrifuging the digested mixture and then collecting the cardiorespiratory progenitors;
SC) performing hanging drop culture on the cardiorespiratory progenitors for 46-52 hours by inducing embryoids to obtain a first suspension, with 1,970-2,030 cells in each 15 microliters (μL) of the first suspension;
SD) performing suspension culture on the first suspension, comprising:
culturing the first suspension in a differentiation medium for 22-26 hours (h) to obtain a first cultured product, then culturing the first cultured product in a basic medium for 22-26 h to obtain a second cultured product, digesting the second cultured product into a single-cell suspension, and inoculating the single-cell suspension on a Petri dish coated with gelatin to obtain inoculated cells, wherein the basic medium is a Roswell Park Memorial Institute 1640 medium (RPMI 1640 medium) added with 2 mM L-glutamine, 1% NEEA, 1% penicillin/streptomycin and 0.1 mM β-mercaptoethanol; the differentiation medium is the basic medium added with 12 μM CHIR-99021; and
SE) continuing to culture the inoculated cells with the ABC medium, changing the ABC medium every two days, and subculturing until a cell confluence is more than 90%.

5-10. (canceled)
